(19) United States
(12) Patent Application Publication
(10) Pub. No.: US 2010/0086491 A1
(43) Pub. Date: Apr. 8, 2010

(54) Capsule Used for Measuring Flow Information Using X-Rays and Method of Measuring Flow Information Using the Same

(75) Inventors: Sang-Joon Lee, Pohang-city (KR); Guk-Bae Kim, Seongbuk-gu (KR); Nam-Yun Lim, Gimhae-shi (KR)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: POSTECH ACADEMY-INDUSTRY FOUNDATION, Pohang -city (KR)

(21) Appl. No.: 12/275,391

(22) Filed: Nov. 21, 2008

(30) Foreign Application Priority Data
Oct. 8, 2008 (KR) 10-2008-0098751

(51) Int. Cl. A61K 49/04

(52) U.S. Cl. 424/9.44; 424/9.4

(57) ABSTRACT

The present invention relates to a capsule used for measuring flow information using X-rays and a method of measuring flow information using the same. More particularly, the present invention relates to a capsule used for measuring flow information using X-rays including a biocompatible polymer, an organic contrast agent or deionized water, and a cross-linking agent, wherein the biocompatible polymer that is cross-linked with the cross-linking agent is filled with an organic contrast agent or is empty. Thereby, it is possible to remarkably increase the image-capturing time, to accurately and quantitatively measure the blood flow, and to gain accurate in vivo flow information.

### CROSS-REFERENCE TO RELATED APPLICATION

This application claims priority to and the benefit of Korean Patent Application No. 10-2008-0098751 filed in the Korean Intellectual Property Office on Oct. 8, 2008, the entire contents of which are incorporated herein by reference.

### BACKGROUND OF THE INVENTION

(a) Field of the Invention

The present invention relates to a capsule used for measuring flow information using X-rays and a method of measuring flow information using the same. More particularly, the present invention relates to a capsule used for measuring flow information using X-rays that is capable of increasing imaging time, accurately and quantitatively measuring blood flow, and gaining accurate flow information inside a living body, and a method of measuring flow information using the same.

(b) Description of the Related Art

Recently, hemodynamics has drawn large attention as a main factor of cardiovascular diseases, so requirements of fusing the medical and engineering fields have increased. As abnormal blood flow such as formation of recirculation flow or low-shear-stress region is considered as a pathogenic mechanism of cardiovascular diseases, accurate quantitative information on blood flow is required.

X-rays have been widely used for medical diagnosis and nondestructive inspection since they can easily transmit through opaque bodies or materials. Particularly, since synchrotrons and digital image processors have been developed, it is possible to provide images of living samples in high spatial resolution and with an excellent contrast ratio. Therefore, new imaging technologies using X-rays have been developed and applied to various fields such as bio-science, medical engineering, and material engineering. X-ray particle image velocimetry (PIV), which can measure quantitative velocity field information of opaque flow, is one of them. Particle image velocimetry is a quantitative flow visualization technique that has been widely used in the hydrodynamics field, and it obtains velocity field information by applying a digital image processing to a flow image including tracer particles.

Conventionally, the X-ray image processing mainly uses a liquid iodine-based contrast agent or a barium-based contrast agent. Particularly, a biocompatible polymer has been used by suspending it in an iodine-based contrast agent or a barium-based contrast agent. However, if such contrast agent is used, it may cause several problems such as unstable suspension due to a specific gravity difference between the biocompatible polymer and the contrast agent, bio-safety of the iodine compound, and a short imaging time.

When such a liquid contrast agent is mixed with a fluid that is to be measured, the liquid agent in an X-ray image is not appeared as distinguished particles. Therefore, it is impossible to apply it as a tracer particle for flow analysis to determine quantitative flow information.

### SUMMARY OF THE INVENTION

One embodiment of the present invention provides a particle-shaped capsule used for measuring flow information using X-rays that has a long imaging time and that accurately measures a blood flow quantitatively.

Another embodiment of the present invention provides a method of obtaining flow information of a blood flow using the capsules as tracer particles.

The embodiments of the present invention are not limited to the above technical purposes, and a person of ordinary skill in the art can understand other technical purposes.

According to one embodiment of the present invention, provided is a capsule used for measuring flow information using X-rays that includes a biocompatible polymer, an organic contrast agent, and a cross-linking agent.

According to another embodiment of the present invention, provided is a capsule used for measuring flow information using X-rays that includes a biocompatible polymer, deionized water, and a cross-linking agent.

According to a further embodiment of the present invention, provided is a method of measuring flow information using a capsule as a tracer particle.

Further embodiments of the present invention will also be described in detail.

According to one embodiment of the present invention, it is possible to provide a capsule used for measuring flow information using X-rays with a much longer image-capturing time, compared to that of the conventional liquid contrast agent, to measure in vivo flow, which is invisible to the naked eye, with several-micrometer accuracy, and to measure a real-time velocity distribution variation of opaque flows. Accordingly, it is possible to make a landmark turning point in the medical field as well as to realize early diagnosis of circulatory diseases.

### BRIEF DESCRIPTION OF THE DRAWINGS

FIG. 1 shows a capsule used for measuring flow information using X-rays according to one embodiment of the present invention.

FIG. 2 is a flowchart showing a process of making capsules used for measuring flow information using X-rays.

FIG. 3 illustrates microfluidic technique, which is one method of making capsules used for measuring flow information using X-rays according to one embodiment of the present invention.

FIG. 4 illustrates a fundamental principle of particle image velocimetry, which is one method of measuring flow information using X-rays according to one embodiment of the present invention.

FIG. 5 shows a scanning electron microscope (SEM) image of the capsule used for measuring flow information using X-rays according to Example 1.

FIG. 6 is a graph showing energy dispersive spectroscopy (EDS) analysis results on constituting materials (central part) in the capsule used for measuring flow information using X-rays according to Example 1.

FIG. 7 is a graph showing energy dispersive spectroscopy (EDS) analysis results on constituting materials (central part) in the capsule used for measuring flow information using X-rays according to Example 2.

FIG. 8 shows an X-ray image of capsules including organic contrast agents according to Example 1.

FIG. 9 shows a contrast ratio graph of the capsule including organic contrast agents according to Example 1.

FIG. 10 shows an X-ray image of the capsule including no organic contrast agents according to Example 2.

FIG. 11 shows a contrast ratio graph of a capsule including no organic contrast agents according to Example 2.

### DESCRIPTION OF REFERENCE NUMERALS INDICATING PRIMARY ELEMENTS IN THE DRAWINGS

| | |
|---|---|
| 1: capsule used for measuring flow | |
| information using X-rays | |
| 11: exterior wall material | 12: interior wall material |
| 2: microfluidic device | 21: flow path of liposoluble |
| | solution |
| 22: flow path of a mixture including | |
| water-soluble solution or water-soluble | |
| solution, and biocompatible polymer | |
| 23: capsule used for measuring flow | |
| information using X-rays | |

### DETAILED DESCRIPTION OF THE INVENTION

Exemplary embodiments of the present invention will hereinafter be described in detail. However, these embodiments are only exemplary, and the present invention is not limited thereto.

A capsule used for measuring flow information using X-rays according to one embodiment of the present invention includes a biocompatible polymer, an organic contrast agent, and a cross-linking agent. The inside of the biocompatible polymer, which is cross-linked by a cross-linking agent, is filled with the organic contrast agent.

The capsule used for measuring flow information using X-rays according to another embodiment of the present invention includes a biocompatible polymer, deionized water, and a cross-linking agent. The inside of the biocompatible polymer, which is cross-linked by a cross-linking agent, is empty.

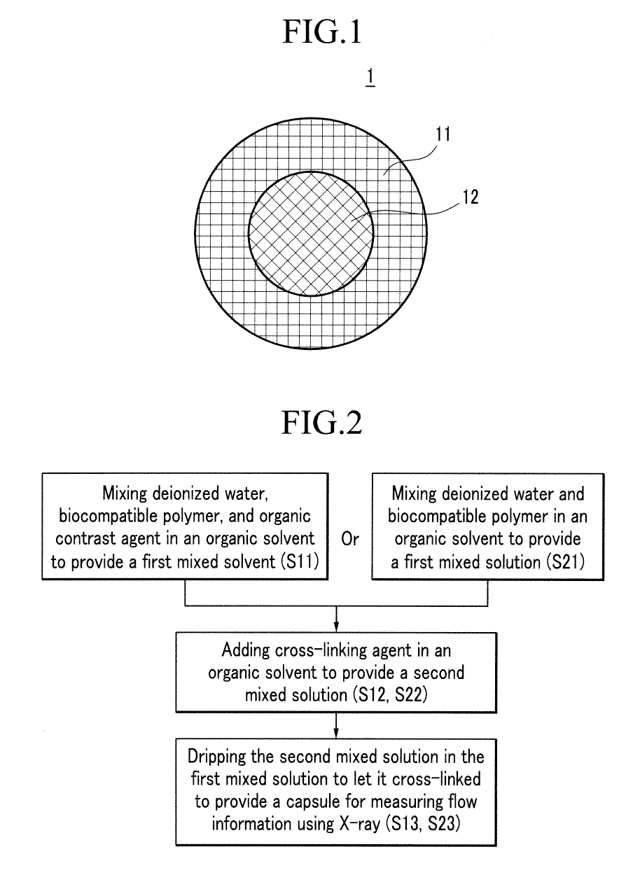

The shape of the capsule 1 used for measuring flow information using X-rays is not limited, but according to one embodiment, the biocompatible polymer is completely covered on the surface of the capsule 1 as an exterior wall material 11, as shown in FIG. 1. The capsule 1 includes an interior wall material 12 partially including a biocompatible polymer cross-linked by cross-linking agents, or having an organic contrast agent or empty space.

In addition, the organic contrast agent may include any material used in this field, but according to one embodiment, it is selected from the group consisting of an iodine-based organic contrast agent, a barium-based organic contrast agent, and a mixture thereof.

Particularly, the organic contrast agent includes iodine-based organic contrast agents selected from the group consisting of metrizamide, diatrizoate, ioxaglate, iopentol, iopamidol, iomeprol, iotrolan, iohexol, ioversol, ioxilan, iopromide, iodixanol, lobitridol, and mixtures thereof. In one embodiment, one selected from the group consisting of iopamidol, iomeprol, iodixanol, and mixtures thereof may be appropriate.

On the other hand, when the capsule according to one embodiment has a hollow inside that is filled with a biocompatible polymer cross-linked with cross-linking agents, which is filled with pores, it is more preferable to measure micro-scale flow information using clinical X-rays or synchrotron X-rays. Particularly, with X-ray micro-imagery using synchrotron X-rays, it is possible to obtain a higher resolution image than with clinical X-rays.

When a hollow capsule filled with pore is used for a capsule for measuring flow information using X-rays, it generates a difference of refractive index at the interface between a gas layer corresponding to pore and a solid layer corresponding to the biocompatible polymer. Due to difference in refractive indexes, the irradiated X-ray beam is concentrated to the interface, so it provides merits in that the gas layer corresponding to pore of the capsule appears brighter, and the profile also becomes sharper at the boundary thereof.

The biocompatible polymer is any conventionally used biocompatible polymer, and its examples include, but are not limited to: polyalkylenevinylalcohols such as polyvinylalcohol (PVA) and polyethylenevinylalcohol; polylactic acid; polylactide glycolide; polyalkyleneoxides such as polyethylene oxide; cellulose acetate; poly(meth)acrylate; polyalkylene-vinylacetates such as polyethylene-vinylacetate; polyvinylpyrrolidone; polycaprolactone; polyhydroxyalkyl(meth)acrylates such as polyhydroxyethyl(meth)acrylate; collagen; gelatin; keratin; alginate; alginic acid; chitin; chitosan; and mixtures thereof. In some embodiments, biocompatible polymers selected from the group consisting of polyvinylalcohol (PVA), polylactide glycolide (PLGA), and mixtures thereof may be preferable. As used herein, the term “alkylene” refers to a C2 to C20 alkylene, and preferably a C2 to C10 alkylene, and the term “alkyl” refers to a C1 to C20 alkyl, and preferably a C1 to C10 alkyl.

The capsule according to one embodiment of the present invention includes a cross-linking agent. The cross-linking agent may include any cross-linking agents used in this field, but according to one embodiment, it includes glutaraldehyde.

The capsule according to one embodiment has a particle diameter ranging from 0.4 to 100 μm. According to another embodiment, it ranges from 0.5 to 80 μm. When the particle diameter of the capsule is below the range, it is impossible to obtain a particle image due to the limitation of spatial resolution, or it may be hard to distinguish it from the adjacent tissue due to insufficient X-ray absorption. On the other hand, when the particle diameter is too large, the volume and weight are increased too much to detect a bio-flow such as blood correctly. Therefore, the particle diameter is preferably within the range.

The following Reaction Scheme 1 illustrates capsulation of the capsule used for measuring flow information using X-rays according to one embodiment of the present invention.

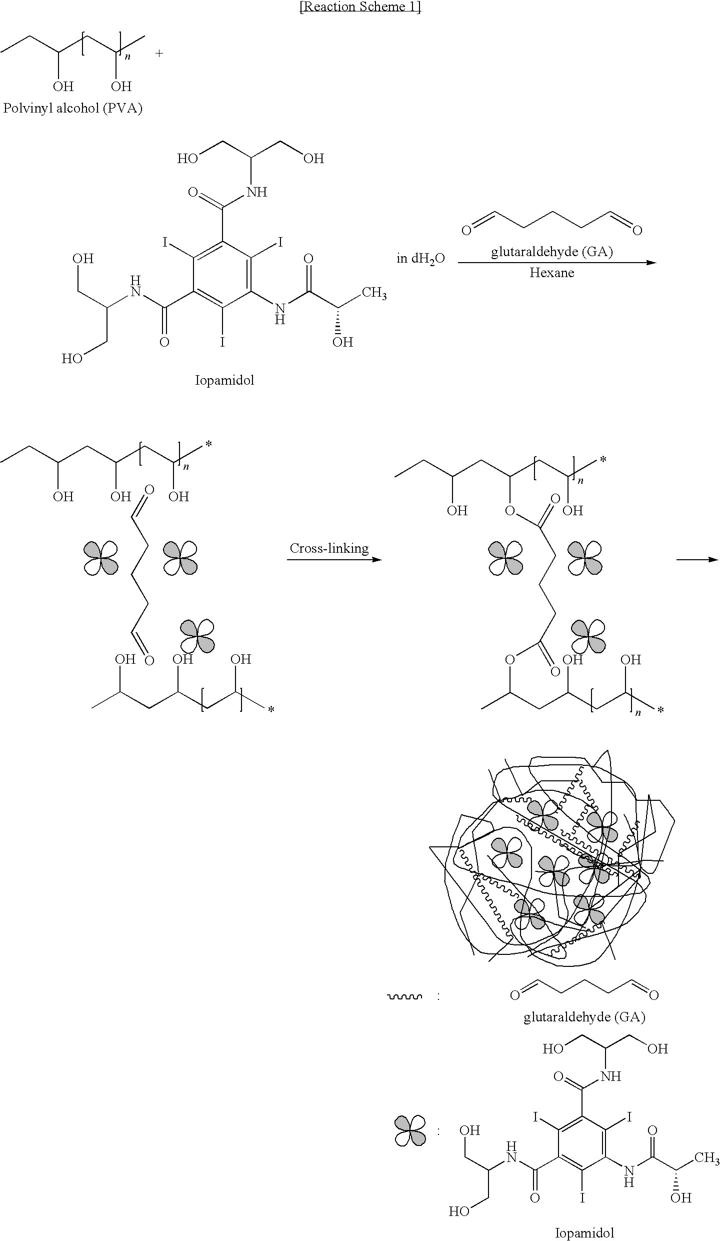

The capsule according to one embodiment of the present invention can be significantly used for determining flow information related to the stomach or cardiovascular system.

The method of making the capsule of the present invention can include any conventionally used method in this field, but the capsule may be manufactured in accordance with the following methods as shown in FIG. 2.

Firstly, the capsule used for measuring flow information using X-rays can be manufactured by: mixing deionized water, a biocompatible polymer, and an organic contrast agent in an organic solvent to provide a first mixed solvent (S11); adding a cross-linking agent to an organic solvent to provide a second mixed solution (S12); and dripping the second mixed solution into the first mixed solution to allow it to cross-link (S13).

Alternatively, the capsule used for measuring flow information using X-rays can be manufactured by: mixing deionized water and a biocompatible polymer in an organic solvent to provide a first mixed solution (S21); adding a cross-linking agent to an organic solvent to provide a second mixed solution (S22); and dripping the second mixed solution into the first mixed solution to allow it to cross-link (S23).

The characteristics such as kind of each constituent, particle diameter, and shape of the obtained capsule are the same as in the description of the capsule used for measuring flow information using X-rays.

Hereinafter, the method of making a capsule used for measuring flow information using X-rays is described in detail.

Firstly, in the step of S11 of mixing deionized water, the biocompatible polymer, and the organic contrast agent in an organic solvent to provide a first mixed solution, the deionized water, biocompatible polymer, and organic contrast agent are mixed at 10 to 30 volume % based on 100 volume % of the organic solvent. In addition, the deionized water, biocompatible polymer, and organic contrast agent are mixed in a weight ratio ranging from 1:10 to 1500:10 to 1500 considering ease of capsulation and X-ray absorption.

On the other hand, in the step of S21 of mixing deionized water and the biocompatible polymer in the organic solvent to provide a first mixed solution, the deionized water is mixed with the biocompatible polymer at 10 to 30 volume % based on 100 volume % of the organic solvent. The deionized water and the biocompatible polymer are mixed in a weight ratio ranging from 1 :10 to 1500 considering the ease of capsulation and X-ray absorption.

The organic solvent may be a generally-used organic solvent such as acetone, methanol, ethanol, isopropyl alcohol, n-propyl alcohol, n-hexane, butanol, dimethyl acetamide (DMAc), dimethyl formamide, dimethylsulfoxide (DMSO), N-methyl-2-pyrrolidone (NMP), tetrahydrofuran (THF), tetrabutylacetate, n-butylacetate, m-cresol, toluene, ethylene glycol (EG), γ-butyrolactone, hexafluoroisopropanol (HFIP), and so on.

In the steps S12 and S22 of adding a cross-linking agent to an organic solvent to provide a second mixed solution, the cross-linking agent is added at 1 to 10 volume % based on 100 volume % of the organic solvent. If the cross-linking agent is added at an insufficient amount, it is difficult to obtain a pertinent capsule; on the other hand, if the cross-linking agent is added at an excessive amount, the capsule wall is too thick to contain sufficient contrast agent, or the boundary between the pore and the capsule wall becomes unclear so as to deteriorate absorption sensitivity of X-rays. Therefore, it is beneficial for the cross-linking agent to be added within the range. The cross-linking agent is added to the mixed solution while stirring so that the cross-linking agent is uniformly mixed in the organic solvent.

In the steps of S13 and S23 of dripping the second mixed solution into the first mixed solution and allowing it to cross-link for providing a capsule that can be used for measuring flow information using X-rays, the cross-linking process is carried out by agitation or microfluidic techniques. In one embodiment, the agitation is carried out under the condition of speed ranging from 300 to 2000 rpm, and in another embodiment, the speed ranges from 400 to 800 rpm. When the agitation speed is too slow, the particle size becomes too large; on the other hand, when it is too fast, the particle size becomes too small. Therefore, it is preferable to maintain the agitation speed within the range. The agitation is carried out at room temperature.

Alternatively, the capsule used for measuring flow information using X-rays may be manufactured by microfluidic techniques.

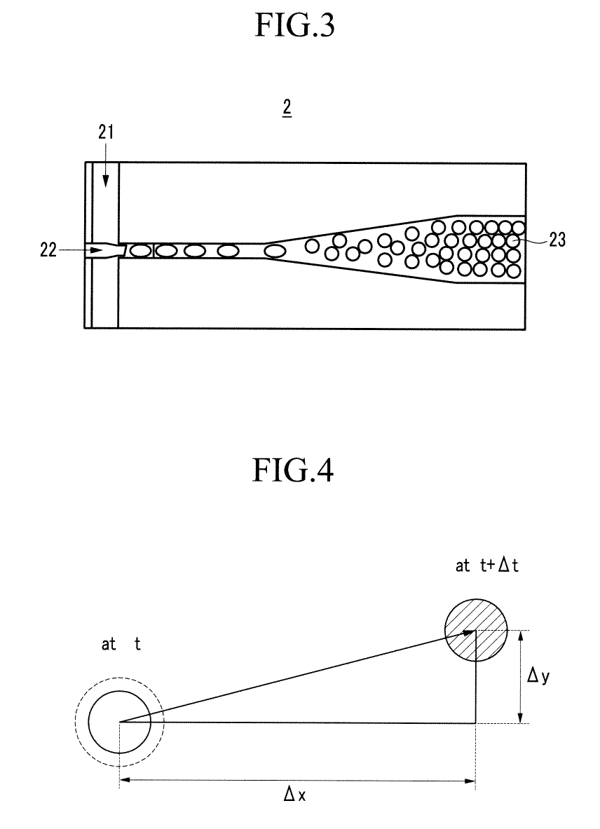

Microfluidic techniques are generally used as a drug delivery system (DDS), and it is also used in fabricating a capsule used for measuring flow information using X-rays according to the present invention. As shown in FIG. 3, a microfluidic device 2 includes several various-shaped channels including a cross channel that are directed into one channel. At the channel, a water-soluble solution flows into the central part (22) of the channel, but a liposoluble solution (for example, a liposoluble polymer or organic solvent) moves from the side part (21) of the channel. During this process, droplets of which the water-soluble solution is present inside the liposoluble solution are formed. It is possible to control droplet size by adjusting the amount of inflow of the liposoluble solution and the water-soluble solution. Then a biocompatible polymer is cross-linked to capsulate the droplet of a micro-size to provide a capsule 23 used for measuring flow information using X-rays.

On the other hand, the capsule used for measuring flow information using X-rays can be manufactured by inflowing the biocompatible polymer along with the water soluble solution flowing into the central part of the channel.

According to another embodiment of the present invention, provided is a method of measuring flow information including the step of using the capsule used for measuring flow information using X-rays as a particle tracer. Particularly, it is possible to obtain in vivo flow information of living bodies in accordance with the method of measuring flow information using X-rays.

The method of measuring flow information using X-rays includes conventionally-used methods in this field and is not limited, but in one embodiment, it may include flow visualization or X-ray particle image velocimetry (PIV).

The particle image velocimetry is a technique for measuring a quantitative velocity field by digital image-processing a flow image having displacement information on flowing particles. The fundamental principle of particle image velocimetry is shown in FIG. 4. The principal of particle image velocimetry using a digital image processing is to provide an instantaneous velocity field by dividing displacement information (Δx, Δy) of tracer particles obtained from two particle images between a certain time interval(Δt) by the time interval(Δt).

Such particle image velocimetry has merits in that it is possible to provide quantitative instantaneous velocity information of whole flow field with excellent spatial resolution. However, since it should obtain particle images of flow with visible rays, both a test model and a working fluid must be transparent.

The X-ray particle image velocimetry overcomes the limitation that particle image velocimetry cannot measure the flow inside an opaque conduit or the flow of an opaque fluid. The X-ray particle image velocimetry is a technique in which X-ray imagery that is capable of visualizing the inside of an opaque material using X-ray transmission is combined with the particle image velocimetry that can measure both flow inside an opaque material such as a human body and flow of an opaque fluid such as blood.

Accordingly, in the capsule used for measuring flow information using X-rays according to one embodiment of the present invention, it is possible to elongate image-capturing time, to determine in vivo flow information, and to measure real-time velocity distribution of a blood flow. Thereby, it is possible to realize the early diagnosis of circulatory diseases and to make an epochal turning-point in the medical imaging field.

Hereinafter, the present invention is illustrated in more detail with reference to examples. However, these are exemplary embodiments of present invention and are not limiting.

### EXAMPLE 1

0.5 g of polyvinylalcohol was added to 5 ml of deionized water and 0.5 g of iopamidol was added to 5 ml of deionized water, which were then mixed together. 10 ml of the obtained mixed solution of deionized water, polyvinylalcohol, and iopamidol was added to 100 ml of n-hexane and agitated at 450 rpm for 30 minutes to provide a first mixed solution.

On the other hand, 5 ml of glutaraldehyde was added to 100 ml of n-hexane and agitated at 450 rpm for 30 minutes to provide a second mixed solution.

The second mixed solution was dripped into the first mixed solution to allow it to cross-link, so it provided capsules for measuring flow information using X-rays having an average particle diameter of around 50 μm.

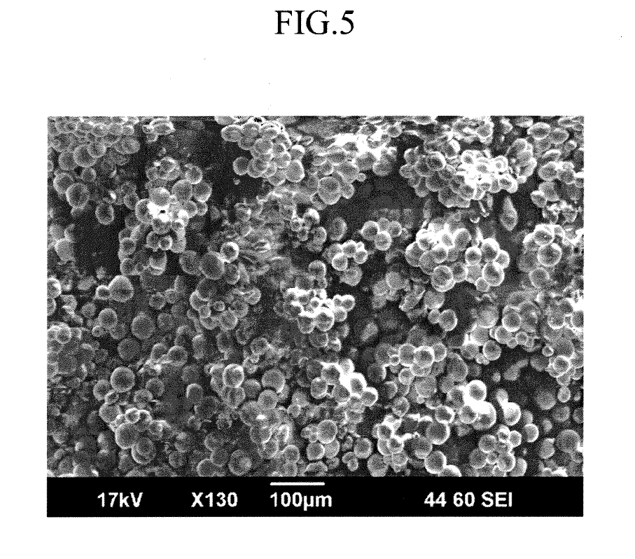

FIG. 5 shows a scanning electron microscope (SEM) image of the obtained capsules. As shown in FIG. 5, spherical-shaped microcapsules having an average particle diameter of around 50 μm were formed.

### EXAMPLE 2

Polyvinylalcohol was added to deinonized water at a mixing ratio of 0.5 g to 5 ml. Then 10 ml of the obtained mixed solution of deionized water and polyvinylalcohol with the above mixing ratio was added to 100 ml of n-hexane and agitated at 450 rpm for 30 minutes to provide a first mixed solution.

On the other hand, 5 ml of glutaraldehyde was added to 100 ml of n-hexane and agitated at 450 rpm for 30 minutes to provide a second mixed solution.

The second mixed solution was dripped into the first mixed solution to allow it to cross-link to provide a capsule for measuring flow information using X-rays having an average particle diameter of around 50 μm.

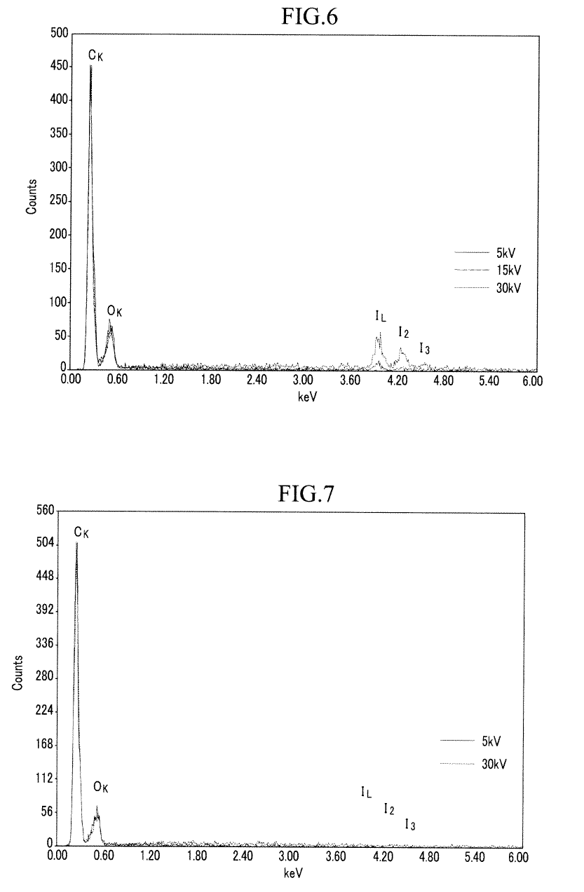

FIGS. 6 and 7 show energy dispersive spectroscopy (EDS) analysis results of capsules for measuring flow information using X-rays according to Examples 1 and 2, respectively. FIG. 6 shows that an iodine component was detected in the capsule fabricated by adding iopamidol; on the other hand, FIG. 7 shows that an iodine component was not detected in the capsule fabricated without adding iopamidol. Thereby, it is confirmed that iopamidol was capsulated.

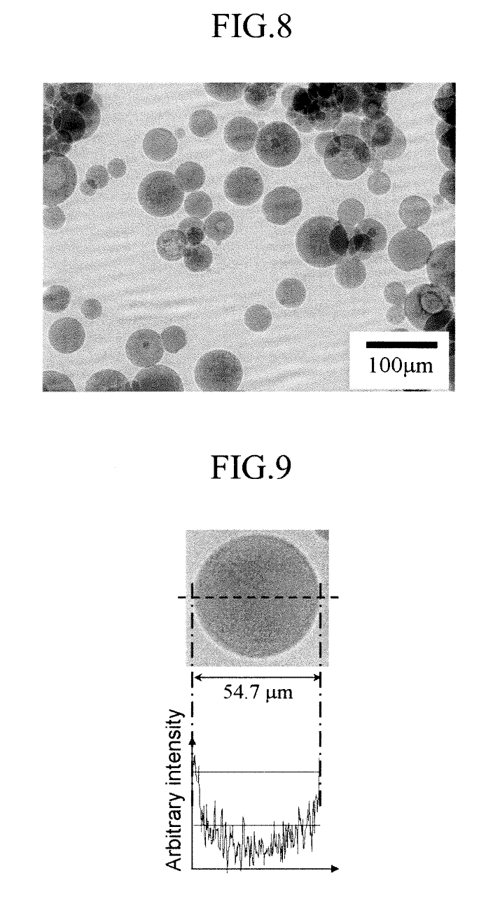

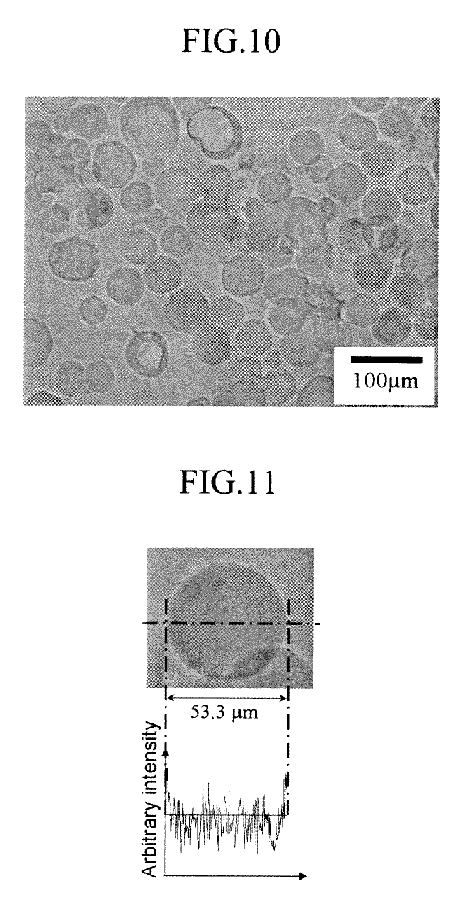

Furthermore, FIGS. 8 to 11 show X-ray images of capsules including an organic contrast agent according to Example 1 (FIGS. 8 and 9) and X-ray images of capsules including no contrast agent according to Example 2 (FIGS. 10 and 11). As shown in FIGS. 8 to 11, it is understood that capsules for measuring flow information using X-rays according to Examples 1 and 2 had excellent contrast ratios by X-rays. Particularly, the capsule including the organic contrast agent according to Example 1 absorbed more X-rays than the capsule without organic contrast agent, so the capsule had less light intensity. This indicates that it had a more improved contrast ratio, so it is possible to measure in vivo flow more accurately.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

1. A capsule used for measuring flow information using X-rays, comprising a biocompatible polymer, an organic contrast agent, and a cross-linking agent.

2. The capsule of claim 1, wherein the inside of the biocompatible polymer, which is cross-linked by the cross-linking agent, is filled with the organic contrast agent.

3. The capsule of claim 1, wherein the organic contrast agent is selected from the group consisting of an iodine-based organic contrast agent, a barium-based organic contrast agent, and mixtures thereof.

4. The capsule of claim 1, wherein the organic contrast agent is selected from the group consisting of metrizamide, diatrizoate, ioxaglate, iopentol, iopamidol, iomeprol, iotrolan, iohexol, ioversol, ioxilan, iopromide, iodixanol, lobitridol, and mixtures thereof.

5. The capsule of claim 1, wherein the biocompatible polymer is selected from the group consisting of polyvinylalcohol (PVA), polyalkylenevinylalcohol, polylactic acid, polylactideglycolide, polyalkylene oxide, celluloseacetate, poly(meth)acrylate, polyalkylene-vinylacetate, polyvinylpyrrolidone, polycaprolactone, polyhydroxyan alkyl(meth)acrylate, collagen, gelatin, keratin, alginate, alginic acid, chitin, chitosan, and mixtures thereof.

6. The capsule of claim 1, wherein the cross-linking agent is glutaraldehyde.

7. The capsule of claim 1, wherein the capsule has a particle diameter ranging from 0.4 μm to 100 μm.

8. A capsule used for measuring flow information using X-rays, comprising a biocompatible polymer, deionized water, and a cross-linking agent.

9. The capsule of claim 8, wherein the inside of the biocompatible polymer, which is cross-linked by the cross-linking agent, is empty.

10. The capsule of claim 8, wherein the biocompatible polymer is selected from the group consisting of polyvinylalcohol (PVA), polyalkylenevinylalcohol, polylactic acid, polylactideglycolide, polyalkyleneoxide, celluloseacetate, poly(meth)acrylate, polyalkylene-vinylacetate, polyvinyl pyrrolidone, polycaprolactone, polyhydroxyan alkyl(meth)acrylate, collagen, gelatin, keratin, alginate, alginic acid, chitin, chitosan, and mixtures thereof.

11. The capsule of claim 8, wherein the cross-linking agent is glutaraldehyde.

12. The capsule of claim 8, wherein the capsule has a particle diameter ranging from 0.4 μm to 100 μm.

13. A method of measuring flow information using x-rays including applying the capsule used for measuring flow information using X-rays according to claim 1 as a particle tracer.

14. The method of claim 13, wherein the method of measuring flow information using X-rays comprises flow visualization or X-ray particle image velocimetry (PIV).

15. The method of claim 13, wherein the method of measuring flow information using X-rays obtains flow information in a living body.
